(19) United States
(12) Patent Application Publication
(10) Pub. No.: US 2016/0000323 A1
(43) Pub. Date: Jan. 7, 2016

(54) MOBILE DEVICE-EMBEDDED SYSTEM AND APPARATUS FOR PROVIDING REAL-TIME AUTOMATED HEALTH DIAGNOSIS BASED ON IRIS SCANNING ANALYSIS

(71) Applicant: Makor Issues and Rights Ltd., Jerusalem (IL)

(72) Inventor: David MYR, Jerusalem (IL)

(21) Appl. No.: 14/320,822

(22) Filed: Jul. 1, 2014

(51) Int. Cl. A61B 3/12; A61B 3/14

(52) U.S. Cl. CPC A61B 3/1216; A61B 3/14

(57) ABSTRACT

The presented invention discloses, in the first aspect, a mobile-device embedded system and apparatus for personalized real-time instant diagnostics of a patient, such a diagnostics produced without human involvement, by scanning a patient's iris with a mini iridology camera, such a camera embedded into the patient' mobile device, and by performing automated real-time iridology analysis by cross-referencing the iridology scan results with diseases data, while iridology analysis package will be pre-installed into the patient' device, and by producing diagnosis if the iridology scan produces results consistent with iris signs indicating the disease. Patient' treatment is recommended by automatically cross-checking a list of diagnosed diseases with the list of treatments. Emergency medical response may be provided automatically to the patient location as determined by GPS/wireless means.

### FIELD OF THE INVENTION

The present invention relates to iridology analysis and, more particularly, to a mobile device based system and apparatus for real-time automated diagnostics of the patient through iris scanning and analysis.

### BACKGROUND OF THE INVENTION

Iridology is an alternative medicine practice for diagnosing human health condition through iris image analysis. The methods used to process a iris image are Canny Edge Detection to detect a specific area on the iris image, Texture Future Analysis to analyze the specific area of iris, and Artificial Neural Network to learn and give health prediction. According to the recent studies, the diagnosis in iridology can be very accurate: up to 95% for accuracy in heart diseases diagnostics, 90% for lung diseases, 90% for spleen, and 90% for liver.

The iris is connected to every organ of the body through the brain and central nervous system through the optic nerve. Because there are many nerve endings in the eye, all linked to the body's organs, iridology assert that examination of the eye, especially the iris, indicates dysfunctions in other organs of the body.

The iris is like a map of the body, showing inherited dysfunctions and pinpointing weaknesses. Changes in the iris reflect changes in the body.

Currently, there are a few computer-based applications available for iridology analysis to substitute manual iris check method. Aigal is a system specialized in iris analysis without producing iris diagnosis. It is based on Bernard Jensen's topology. It enables to save the patient's medical history in a very ordered way. It also allows us to keep track of the patient's evolution throughout the treatment. This system is aimed at collaborating with the iridologist, helping locate the signs in the iris, without making early presumptions or diagnosis. The system automatically displays the organ and the corresponding sector according to Jensen's topology. Once the analysis is completed, the corresponding report can be emitted.

Iridology Analysis Professional—is an additional iridology analysis software currently available. Iridology Analysis Professional provides examination of the pupil, collarette and iris sectors, with several options of iridological topographic charting systems, precisely placed for each and every unique human eye.

Iridology Station 5.1 is a computerized iridology analysis and reporting software allowing users to perform the iridology analysis, manage their iridology images, overlay iridology charts, record client details, scan the iris for color intensity and create reports ready for printing.

Iris Analysis Report software provides an iridology classification, describing detailed iris signs 33 Constitutional Types & Sub-Types, Health Inclinations for each type, Body Systems and Nutrition & Dietary Information.

U.S. Pat. No. 6,425,860 by Sadkhin titled “Therapeutic and diagnostic method and apparatus” discloses a method for treating a patient comprises selecting a zone of a patient's iris to be treated, monitoring the patient's eye to determine a position of the selected zone, and directing energy of a predetermined type and a predetermined magnitude toward the zone. The iris zone is selected in part by automatically scanning the patient's iris to generate a signal encoding patterns in the iris, and automatically comparing pattern data in the signal with previously stored pattern data to derive diagnostic information pertaining to the individual patient. The diagnosis and treatment selection are made by a computer programmed with statistical data correlating iris patterns of hue and light or darkness levels with physiological states of different organs and with possible treatments to counter undesirable conditions.

US Patent Application 20090027618 by Perner titled “Method and Arrangement for Automatic Detection and Interpretation of the Iris Structure for Determining Conditions of a Person” discloses a method for automatic detection and interpretation of the iris structure for determining a condition of a person, two-dimensional recording and digitization of an eyeball with iris is realized by a recording device connected to a data processing system by generating the topography of the iris by dividing the image of the iris into images of circular ring sections and determining features for the circular ring sections and/or of objects in the images of the circular ring sections. Further, correlation is made of the determined objects to body regions in accordance with the topography of the iris are performed.

### REFERENCES CITED

U.S. Pat. No. 6,425,860

US Patent Application 20090027618

### BRIEF DESCRIPTION OF THE DRAWINGS

FIG. 1 provides a general overview of the invented iridology diagnosis and response system.

FIG. 2 shows the major hardware and software components of the invented system and apparatus.

FIG. 3 is a schematic representation of the bi-directional communication data flow in the invented system.

### DETAILED DESCRIPTION OF THE INVENTION

Unlike the abovementioned iridology software and US patent prior art data, in which the iridology-based diagnostics is made by the medical specialist interpreting signals produced by iridology software, our application discloses method for automatic iridology diagnostics performed instantly and without human involvement, with no need in diagnosis and interpretation made by the medical specialist, as well as the novel mobile-based device especially configured for the iridology needs with a special mini iridology camera, and software and hardware means for iridology diagnostics without human involvement.

In the following discussion, numerous specific details are set forth to provide a thorough understanding of the present invention.

Other features and advantages of the present invention will be apparent from the following description taken in conjunction with the accompanying drawings, in which like reference characters designate the same or similar parts throughout the figures thereof.

The invention discloses a mobile-device embedded system and apparatus for personalized real-time medical diagnosing of a patient, such a diagnosing performed instantly and without human involvement, by automatically scanning a patient's iris to generate a signal encoding patterns in the iris, and automatically comparing pattern data in the signal with previously stored individual pattern data to derive diagnostic information pertaining to the individual patient and generating alert signals representative of certain abnormalities of the patient according to individual alert parameters adjusted for every patient enrolled.

We have developed an automatic real-time iris-analysis based mobile device embedded system and apparatus for predicting a person health through analysis of iris image. The system and apparatus include high quality iridology mini camera embedded into the mobile device to enable high quality iris scanning and an automated iridology analysis software package for iridology scan automated analysis.

The system and apparatus works on the majority of operating systems, such as Android, Iphone, Windows Mobile, etc.

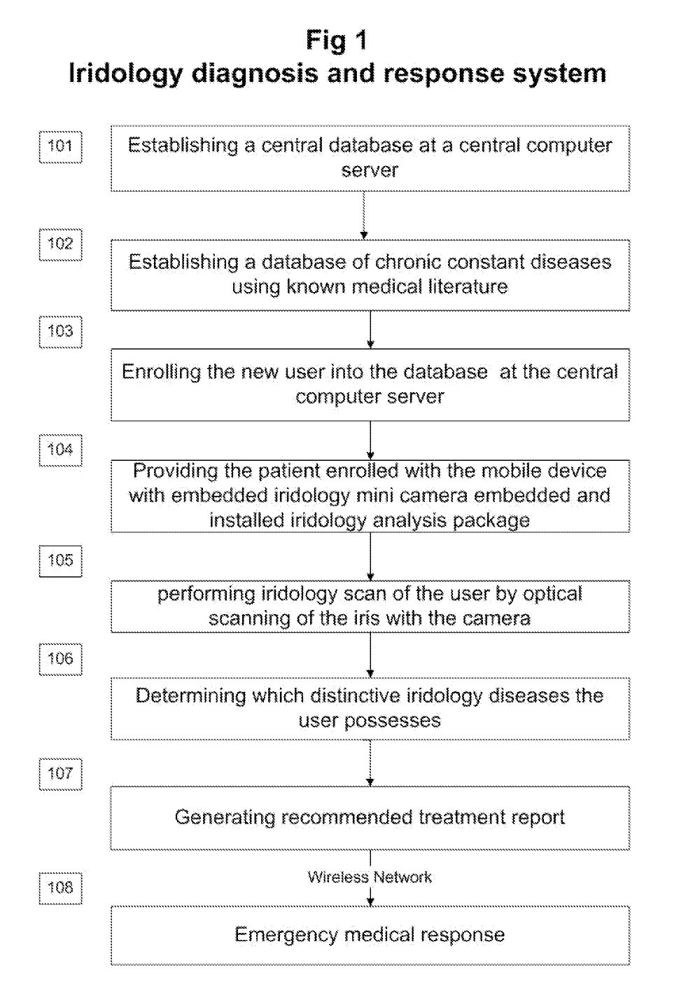

FIG. 1 provides a general overview of the invented iridology diagnosis and response system. To implement the invented system, a central database (101) will be established on a central computer server where personal medical records for each patient will be stored, and to where all iridology data and alerts will arrive.

Such centralized database will be available for viewing, monitoring and analysis both on-location and through the Internet. For the Internet use, a special web-based portal will be developed.

An extensive statistical work will be applies to analyze such data for each patient separately and for all patients together as certain statistical averages and other statistics will be created.

For that, the system will cross-check a list of treatments for the diagnosed diseases, will be installed into the database by comparing the diagnosed disease with the list of treatments available.

A database with a number of chronic constant diseases (102) will be created and established on the abovementioned server, such diseases will be determined using known medical literature.

For each patient enrolled, a new medical file in a medical dispatcher center will be created (103). This medical file of the patient will include a full medical history of the patient, history of surgery operations and procedures, normal levels of psychological and cardiovascular parameters, such as pulse, blood pressure, etc. In particular, the patient medical record will include personal thresholds to determine if the event is irregular for the particular patient.

Information on iridology, reports, and alerts will be provided to the patient's personal file and to the centralized database through wireless communication. Iridology scans data, abnormalities alerts data, medical response to the alert data, as well as alert treatment data and results of alert treatment (medical condition after the treatment) will also be provided to the patient's personal file and to the centralized database through wireless communication means.

Every patient enrolled into the disclosed system receives/acquires the mobile device with the said high quality iridology mini camera embedded into it, and iridology analysis package pre-installed into it (104).

Therefore, the apparatus of the current invention is capable of bi-directional transmittance of information in a wireless phone network. In one embodiment of the invention, the iridology analysis software will be built-in as a chip on the board of such a mobile device.

Following user enrollment and receiving the abovementioned apparatus 104, the user will be able to perform iridology self-diagnosis; such self-diagnosis will be performed without human enrollment.

In order to perform the abovementioned diagnosis without human involvement, the user has to pushing the special button associated with the self-diagnosis on the invented device 104. Such a button might be embedded as a hardware on the invented device or programmed as a button on a touch screen of the mobile device.

The said diagnosis without human enrollment will be performed by pointing the apparatus 104 to his eyes and performing iridology scan, such scan is made with iridology mini camera, embedded into the apparatus to provide high quality of the scan by pointing the mini camera (105). The 104 apparatus of the current invention further comprises a scanner for automatically scanning the patient's iris to generate a signal encoding patterns in the iris. Iridology diagnostic software is operatively connected to the iris scanner for automatically comparing iridology diagnostics results data with previously stored pattern data to derive diagnostic information pertaining to the individual patient. The diagnostic component is operatively connected to the energy applicator for at least partially controlling operation thereof.

On the next step, the iridology analysis software will automatically analyze (106) the iris scan image obtained. The iridology analysis in general reveals body constitution, inherent weaknesses, and levels of health and transitions that take place in a person's body according to the way one lives.

The commercially available iridology analysis packages provide diagnosis of most major diseases: cardiovascular and cerebrovascular, gastrointestinal function, liver function, gallbladder function, kidney function, lung function, brain nerve, bone disease, diabetes, lungs function, blood sugar, human toxin, immune system, gynecology, breast, prostate, etc.

The commercially available iridology analysis packages analyze and provide conclusions mainly from the pupil of the eye, especially from the pupillary and collarette (autonomic nerve wreath) parameters of the human eye which offers high accuracy in detecting ‘active’ pathologies. These programs also consider iris density and other iris factors. Pupil reflexes play the primary role in making a diagnosis of many neurological diseases. Changes in the pupil of color, dimensions, shape, position of center, equality and reflector reactions all have clinical significance. Morphologic signs are important to determine chronic pathologies.

Commercially available iridology software packages are: Iridology Analysis Professional, Aigal, Iris Analysis Report software, Iridology Station 5.1, etc.

The diagnostic information obtained may be used to determine a recommended treatment of the patient. For that, the system will cross-check a list of treatments for the diagnosed diseases, with the diagnosed disease with the list of treatments available. Accordingly, a treatment report will be automatically generated by the invented system (107).

The diagnostic information derived by the scanning of the patient's iris may be printed out for inspection, storage or any other purpose. The treatment report will be carried in the form usable for understanding by the user (no need in medical professional knowledge).

In case, where a dangerous life-threatening disease is diagnosed, a real-time medical response such as sending an ambulance will be automatically generated by the medical emergency center, in one embodiment of the invention, according to those automatically detected signals in accordance with abnormalities classification (108). Actually, each abnormality alert will be classified according to the severity of the disease alerted. When the abnormality detected justifies an urgent alert, the device will use the cellular network to transfer the emergency signal to the medical center. There the signals will be attended by the medical dispatcher. This will allow the dispatcher working in the center to send ambulance to the patient according to his location detected by the means of GPS or/and cellular network location.

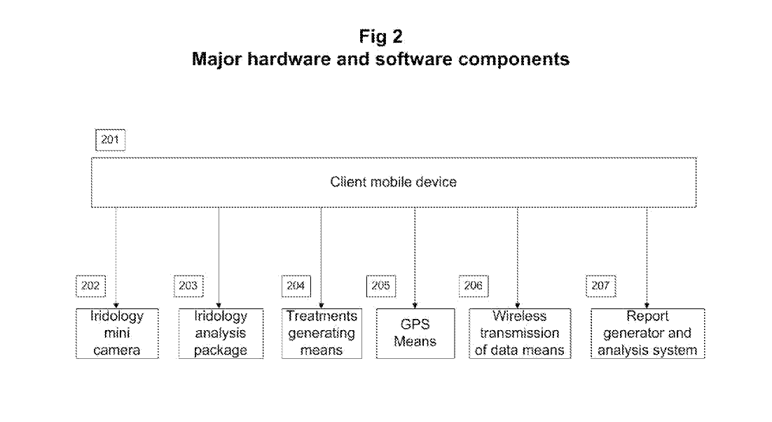

FIG. 2 graphically summarizes major hardware and software components in the invented system and apparatus.

Client mobile device apparatus (201) integrated into the wireless networks includes the following main components:

a) High-quality iridology mini camera (202) will be embedded into the said mobile device apparatus. Such a special camera is necessary for iridology-based scans that can produce high-accuracy diagnostics. The scanning of the patient's iris may be implemented, in accordance with the present invention, by attaching a special iridology mini camera embedded into the mobile phone before the patient eyes. The iridology camera determines specific markings on iris and records user' unique iris shape.

b) Iridology analysis software means (203) will be integrated into the abovementioned mobile device apparatus for real-time automated iridology scan results.

c) Treatment generation means (204) integrated into the abovementioned mobile device apparatus for automatically generating a treatment based on the diagnosis produced.

d) GPS means (205) integrated into the abovementioned mobile device apparatus for determining patient location in real time.

e) Means for wireless transmission of data (206) to the medical emergency center for providing a medical response and to the central computer server for data registration and recording.

f) Report generator means (207) for generating report treatments integrated into the said mobile device. The iris treatment zone may then be selected automatically in response to the diagnostic information. A report with a list of possible treatments to counter undesirable conditions will be printed at the end of the iridology analysis.

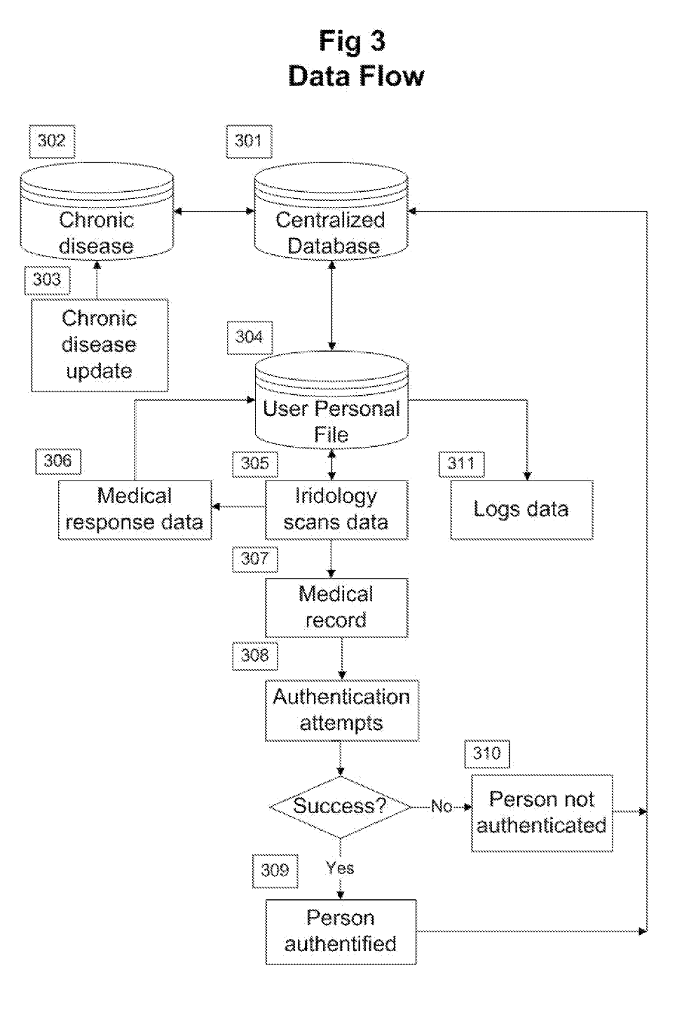

FIG. 3 graphically summarizes data flow in the invented system with regard to person authentication.

As abovementioned, centralized medical database (301) will be established on a central server.

The database 301 will be interactively connected with the chronic diseases database (302) where data is situated regarding constant chronic diseases currently available for diagnosis. Such database will be regularly updated using new medical data (303).

Database with all personal files for all users enrolled (304) will be interactively connected with the central database 301.

The abovementioned user personal files 304 that will include the basic enrollment data pertaining to the user as well as the following data for each user:

Iridology scans files and abnormalities alerts data (305) which includes data on all iridology scans performed by the user. Alerts treatment data and results of alert treatment (medical condition after the treatment) will also be provided to the patient's personal file and to the centralized database through wireless communication.

Medical response data files (306), such data files containing information on medical responses generated as a result of iridology scans 305.

Medical record files (307) with all available diagnosis, including chronic diseases diagnostics, such record files being continuously updated based on the ongoing iridology scanning and diagnostics data.

Biometric authentication attempts data (308) which are based on constant chronic diseases available from the 307. If the authentication attempt is successful, then the person is authenticated (309) and his personal data file is updated accordingly. If the authentication was not successful (310), a non-authenticated attempt will be registered for security purposes.

The user personal file will also contain data on user logs (311) and other miscellaneous information.

Other features and advantages of the present invention will be apparent from the following description taken in conjunction with the accompanying drawings, in which like reference characters designate the same or similar parts throughout the figures thereof.

1. A method for predicting real-time automated person medical diagnosis by performing iridology scans, the method comprising the steps of:
a. maintaining on a central computer server a centralized iridology database which includes respective medical data for a plurality of patients, and a list of medical diseases and dysfunctions which corresponds to iridology data, wherein the medical data includes levels of psychological or cardiovascular parameters associated with each of said patients;
b. providing each of said patients with a mobile device with a mini iridology camera and an iridology analysis package installed therein;
c. performing with the mobile device an iridology scan with the iridology mini camera by pointing the mini camera into eyes of one of the patients,
d. performing automated real-time iridology analysis by cross referencing the iridology scan results with the list of medical diseases and dysfunctions in the central iridology database;
e. producing a respective predicted diagnosis if 1) the iridology scan produces results consistent with any of the medical diseases and dysfunction; and 2) the results when compared with the psychological or cardiovascular parameters maintained in step a) indicates at least one of the medical diseases and dysfunctions;
f. determining, without human involvement, a recommended treatment for the patient for the respective predicted diagnosis by automatically cross-checking a list of treatments for the respective predicted diagnosis in step e with the list of treatments available for the respective predicted diagnosis and providing the respective predicted diagnosis to the patient based on the medical data stored on the central computer server; and
g. generating, without human involvement, a medical abnormality alert if said predicted diagnosis is one of a plurality of life-threatening diseases and providing a real-time medical response by using a cellular network to transfer an emergency signal to a medical center.

2. The method of claim 1, further comprising the step of: h) generating a medical report with patient medical history data and saving this data on a centralized database.

3. The method of claim 1, further comprising the step of: i) storing medical information obtained from the patient at the centralized database.

4. The method of claim 1, further comprising the step of: j) providing a wireless network to facilitate a bi-directional exchange.

5. The method of claim 1, wherein a pre-determining confidence threshold is determined, and the diagnosis is made if the confidence threshold has been exceeded.

6. The method of claim 1, wherein said wireless device is a selected one of a cellular mobile telephone, a multimedia phone or a personal digital assistant (PDA).

7. Apparatus for predicting real-time automated diagnostics of patients by automatically scanning a person's iris, comprising the following elements:
a) a mobile device integrated into a wireless networks;
b) an iridology mini camera included with the mobile device,
c) Iridology analysis software included with the mobile device for performing with the mobile device an iridology scan with the iridology mini camera by pointing the mini camera into eyes of one of the patients to obtain iridology scan results, by cross referencing the iridology scan results with a list of medical diseases and dysfunctions in a central iridology database, and by comparing results of the cross referencing with psychological or cardiovascular parameters maintained for said one of the patients to obtain a respective predicted diagnosis;
d) a treatment generator included with the mobile device for generating a treatment based on the diagnosis obtained, without human involvement, by comparing the respective predicted diagnosis with a list of treatments available for the respective predicted diagnosis and providing said treatment from said list of treatments to the one of the patients;
e) a GPS for determining patient location in real time;
f) Wireless transmitter for transmitting data to the medical emergency center for providing a medical response and to the central computer server for data registration and recording;
g) a report generator for generating report treatments integrated into the mobile device.

8. The method of claim 1, further comprising the step of sending an ambulance responsive to a medical center receiving the medical abnormality alert.

9. The method of claim 1, wherein thresholds are created based on the physiological characteristics, and the predicted diagnosis is generated based on the iridology analysis predicting a condition based on physiological data associated with the iridology scan in step c) exceeding at least one of said thresholds.

10. The method of claim 1, further comprising the step of advising to take medicine responsive to receiving the medical abnormality alert.
